(19) United States
(12) Patent Application Publication
(10) Pub. No.: US 2022/0362117 A1
(43) Pub. Date: Nov. 17, 2022

(54) Effective treatment for dandruff, a composition for treating dandruff and a method for making same

(71) Applicants: Alexander Sepper, New York, NY (US); Dennis Tubian, New York, NY (US); John Navi, New York, NY (US)

(72) Inventors: Alexander Sepper, New York, NY (US); Dennis Tubian, New York, NY (US); John Navi, New York, NY (US)

(21) Appl. No.: 17/744,701

(22) Filed: May 15, 2022

Related U.S. Application Data
(60) Provisional application No. 63/189,444, filed on May 17, 2021.
(60) Provisional application No. 63/189,454, filed on May 17, 2021.

(51) Int. Cl. A61K 8/27; A61Q 5/00; A61K 8/49; A61K 8/46; A61K 8/73; A61K 8/20

(52) U.S. Cl. CPC A61K 8/27; A61Q 5/006; A61K 8/494; A61K 8/492; A61K 8/466; A61K 8/738; A61K 8/20

(57) ABSTRACT

An effective treatment for dandruff and seborrheic dermatitis, a composition for treating dandruff, and a method for making such a composition

### RELATIONSHIP TO OTHER APPLICATIONS

The present non-provisional application claims priority to and the benefit of U.S. application Ser. No. 63/189,444, filed 17May 2021 and U.S. application Ser. No. 63/189,454, filed 17May 2021, both of which are incorporated by reference.

### FIELD OF THE INVENTION

The embodiments of the present invention relate to an effective treatment for dandruff and seborrheic dermatitis, a composition for treating dandruff, and a method for making such a composition.

### BACKGROUND

Dandruff and seborrheic dermatitis are a problem that have confronted the consumer market for many years. There is a long-felt need for an effective treatment. The same anti-dandruff shampoos have been prevailing on the market for many years and there is no real breakthrough in this direction. Additionally, according to epidemiological data, the frequency of this problem is increasing, apparently due to the constantly growing level of industrialization of many aspects of our life, including nutrition, the market for various personal and household hygiene products, which obviously contribute to the development of metabolic and hormonal abnormalities in cellular activity and functions of the cellular elements of the scalp and hair follicles.

Numerous innovations for dandruff treatments have been provided in the prior art, which will be described below in chronological order to show advancement in the art, and which are incorporated herein in their entirety by reference thereto. Even though these innovations may be suitable for the specific individual purposes to which they address, however, they differ from the embodiments of the present invention. For example:

U.S. Pat. Nos. 3,920,835 and 3,984,566 to Van Scott et al. that teach a treatment to alleviate the symptoms of diseases characterized by defects in keratinization including the topical application of an ointment or lotion containing one or more lower aliphatic compounds having from two to about six carbon atoms, and, preferably, having α-carbon functionality. The compounds include α-hydroxy acids, keto acids, and esters thereof, and 3-hydroxybutyric acid. The therapeutic composition may include one or more of the compounds present in a total of from one to twenty percent in either a water or alcohol solution or an ointment. Topical application to affected areas has been found to achieve a complete remission of dandruff, acne, and palmar and plantar hyperkeratosis.

U.S. Pat. No. 4,105,782—issued to Yu et al. on Aug. 8, 1978 in U.S. class 424 and subclass 283—teaches a preventive, as well as therapeutic treatment, to alleviate the symptoms of diseases characterized by defects in keratinization, which includes the topical application of a solution, lotion, cream, or shampoo containing one or more of the α-hydroxy or α-keto acid amides and/or ammonium salts. The compounds include amide and/or ammonium salt forms of citric acid, glycolic acid, glucuronic acid, galacturonic acid, glucuronolactone, gluconolactone, α-hydroxybutyric acid, α-hydroxyisobutyric acid, malic acid, mandelic acid, pyruvic acid, β-phenyllactic acid, β-phenylpyruvic acid, saccharic acid, tartaric acid, tartronic acid, and β-hydroxybutyric acid. The therapeutic composition may include one or more of the compounds present in a total amount of from 1 to 20 percent. Topical application to affected areas has been found to achieve from a substantial to a complete remission of dandruff and acne.

U.S. Pat. No. 4,170,229—issued to Olson on Oct. 9, 1979 in U.S. class 128 and subclass 67—teaches a Vitamin A-containing crystal clear transparent aqueous sprayable emulsions for application to hair and scalp according to a spray-on then brush-in technique that has been found to effectively reduce itching, flaking of common dandruff and seborrhea, condition the hair and scalp, and in some instances reduce excessive falling hair.

U.S. Pat. No. 5,221,533—issued to Perlman on Jun. 22, 1993—teaches a skin lotion and methods of use involving a skin lotion including effective amounts of allantoin, castor oil, oleic acid, calcium hydroxide powder, and witch hazel water. The methods of use include as a keratoplastic and keratolytic for removing the “piled up” hyperkeratotic scales in seborrheic capitis and psoriasis vulgaris, and to expedite the involution of the lesions of acute herpes simplex.

U.S. Pat. No. 6,168,798—issued to O'Halloran et al. on Jan. 2, 2001 in U.S. class 424 and subclass 401—teaches methods and compositions that are mild and nonirritating for reducing and eliminating skin disorders, particularly, blemishes and redness associated with acne. The hydroalcoholic compositions contain a keratolytic compound, preferably, αβ-hydroxy carboxylic acid, more preferably, salicylic acid or pharmaceutically acceptable salt thereof, and a glycerophosphate ester or salt formulated with pharmaceutically acceptable carriers. Preferred glycerophosphate salts for use in the methods and compositions are sodium glycerophosphate and calcium glycerophosphate. Additional anti-irritancy agents are not required in the compositions, as the glycerophosphate salt component uniquely endows the compositions with nonirritant properties. Other components that may be formulated into the compositions include fragrances, colorants, emollients, humectants, and antimicrobial agents.

### BRIEF DESCRIPTION OF THE INVENTION

The invention encompasses (1) an effective treatment for dandruff and seborrheic dermatitis, (2) a composition for treating dandruff and seborrheic dermatitis, and (3) a method for making such a composition.

The composition of the present invention includes a number working ingredients that function synergistically to provide an unexpectedly superior effect, permanently treating the underlying causes of dandruff. The ingredients include (1) a group of ingredients aimed at restoring the architectonics of scalp cells and regulating their metabolism and the production and release of sebum; (2) a group of specific anti-fungal ingredients that contribute to the elimination of a fungal infection from the scalp; and (3) a group of ingredients that perform a detoxification function, which is extremely important, because the graft debris can have destructive functions like a living fungus. Therefore, timely and effective removal and detoxification of debris is an important part of effective treatment.

### BRIEF DESCRIPTION OF THE FIGURES OF THE DRAWING

The figures of the drawing are briefly described as follows:

FIGS. 1A-1F are a diagrammatic flowchart of the method of making the composition for treating dandruff.

### DETAILED DESCRIPTION OF THE PREFERRED EMBODIMENTS

The embodiments of the present invention themselves, however, both as to their construction and to their method of operation, together with additional objects and advantages thereof, will be best understood from the following description of the embodiments of the present invention.

### ANTI-DANDRUFF COMPOSITIONS

One object of the embodiments of the present invention is to provide a composition for treating dandruff, which is a complex, one-of-a-kind biologically active system consisting of two conventional parts: an anti-dandruff biologically active module; and a delivery system. The composition includes a number of groups of synergistically working ingredients: a group of ingredients aimed at restoring the architectonics of scalp cells and regulating their metabolism and the production and release of sebum; a group of specific anti-fungal ingredients that contribute to the elimination of a fungal infection from the scalp; and a group of ingredients that perform a detoxification function, which is extremely necessary because the graft debris can have destructive functions like a living fungus. Therefore, timely and effective removal and detoxification of debris is an important part of effective treatment.

The composition for treating dandruff of the embodiments of the present invention is a complex, one of a kind biologically active system consisting of two conventional parts:

1. An anti-dandruff biologically active module made of three compounds; and

2. A delivery system.

The composition for treating dandruff includes a number of ingredients synergistically working together:

1. A group of ingredients aimed at restoring the architectonics of scalp cells and regulating their metabolism and the production and release of sebum;

2. A group of specific anti-fungal ingredients that contribute to the elimination of a fungal infection from the scalp; and

3. A group of ingredients that perform a detoxification function, which is extremely necessary, because the graft debris can have destructive functions like a living fungus. Therefore, timely and effective removal and detoxification of debris is an important part of effective treatment.

The ingredients of the composition for treating dandruff are integrated into the delivery system, which performs a dual function:

1. Firstly, it stabilizes the structure of the substances integrated into it, and thus, prolongs their activity in a specific way; and

2. Secondly, it delivers the target ingredients to the target tissues.

The ingredients that make up the composition for treating dandruff are incorporated into the delivery system with the observance of specific technological conditions.

The steps for formulation of the invention is as follows. The delivery system is made by preparation of two solutions (Solution #1 and Solution #2) as described herein, and mixing said two solutions to produce the delivery system. The “anti-dandruff biologically active module” comprises a further three ingredients which include Zinc Pyrythione, having fungistatic and bacteriostatic properties; Climbazole, which is a topical antifungal agent; and Melatonin, which is a hormone involved in synchronizing circadian rhythms. The delivery system and the anti-dandruff biologically active module are mixed together to create the formulation of the invention.

### DELIVERY SYSTEM Solution #1

| | RANGE |
|---|---|
| INGREDIENT | (% wt/wt) |
| Dymethylsulfoxide (DMSO) | 4.0-36.0 |
| Hydroxypropyl-b-Cyclodextrin (HPBC) | 0.75-27.0 |

### DELIVERY SYSTEM Solution #2

| | RANGE |
|---|---|
| INGREDIENT | (% wt/wt) |
| Distilled Water | 8.0-46.0 |
| Sodium Chloride | 0.2-4.3 |
| Potassium Chloride | 0.7-2.8 |
| Calcium Chloride | 0.03-5.2 |
| Sodium Bicarbonate | 0.02-6.1 |
| Imidazolidynyl Urea | 0.1-4.5 |

### ANTI-DANDRUFF BIOLOGICALLY ACTIVE MODULE

| | RANGE |
|---|---|
| INGREDIENT | (% wt/wt) |
| Zinc Pyrythione | 0.05-5.7 |
| Climbazole | 1.0-6.0 |
| Melatonin | 0.03-3.7 |

The formulation is made by mixing the above ingredients as described herein.

Although the above ranges (wt/wt) are desirable and preferred, in various embodiments the above ranges may be broader or narrower than those shown or may comprise narrower ranges within the stated ranges, and still provide an effective product. They may be provided in any effective and acceptable concentration. For example, in the final formulation DMSO may be present in a final % wt (or vol) of from 4.0-36.0%, or from 1-50%, 5-30%, 7-25% or 10-20%. HPBC may be present in a final % wt (or vol) of from 0.75-27.0%, or 0.5-40%, 1-20%, 2-15% or 3-10%. All amounts and ranges inherently imply an amount +/−10% or +/−20%.

As is typical in such formulations, distilled water (or non-distilled water) may be provided in any embodiment so as to bring the total volume or mass to 100%. And indeed, other inactive ingredients and carriers may be used for the same purpose, essentially as a volume-bulking agent. So, although water is suggested as being present as % vol between 8.0-46.0, is can be any volume up to 99%, for example 8-90%, 10-80%, 10-70%, 10-60%, 10-50%, 10-30% or 10-20% by volume.

Likewise, Sodium Chloride may be present for example as0.01-5%, 0.5-3% or 1-2%. Potassium Chloride, and Calcium Chloride may have similar ranges. Sodium Bicarbonate and Imidazolidynyl Urea may be present for example as 0.01-10%, 0.1-7%, 0.2-6%, or 0.02-6.1%, or 0.1-4.5%.

The active in ingredients are Zinc Pyrythione (fungistatic and bacteriostatic), Climbazole (antifungal) and Melatonin (a hormone).

Typical concentration ranges in % wt/wt are 0.05-5.7% for Zinc Pyrythione but may be provided in any acceptable concentration such as 0.01-10%, 0.05-10%, 0.1-10%. 0.2-10%.

Climbazolemay be provided in any acceptable concentration such as 0.5-10%, 0.75-10%, 10-10% etc.

Melatonin may be provided in any acceptable concentration such as 0.01-5%, 0.01-5%, 0.02-4% etc.

In other embodiments the therapeutic composition may include one or more of the compounds described herein in a total of from one to forty percent in either a water or alcohol solution or an ointment.

Although distilled water is mentioned throughout, non-distilled water may equally be used.

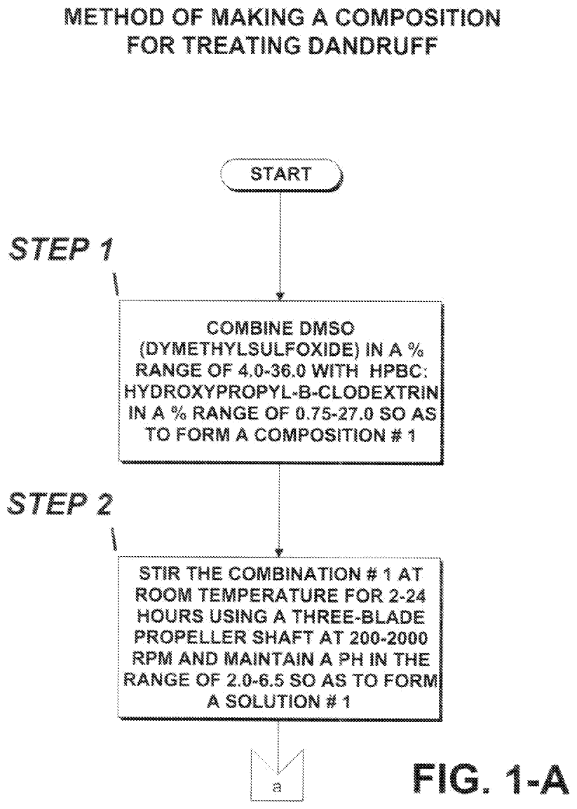

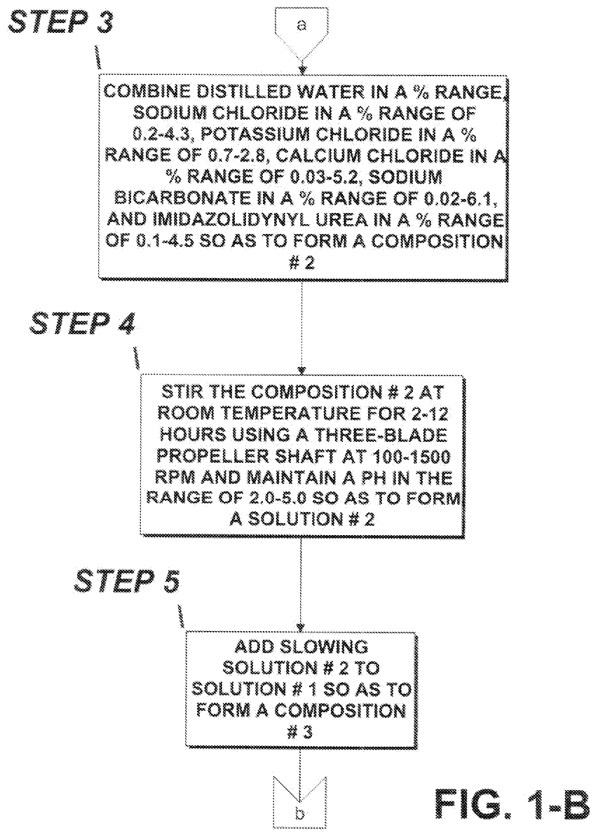

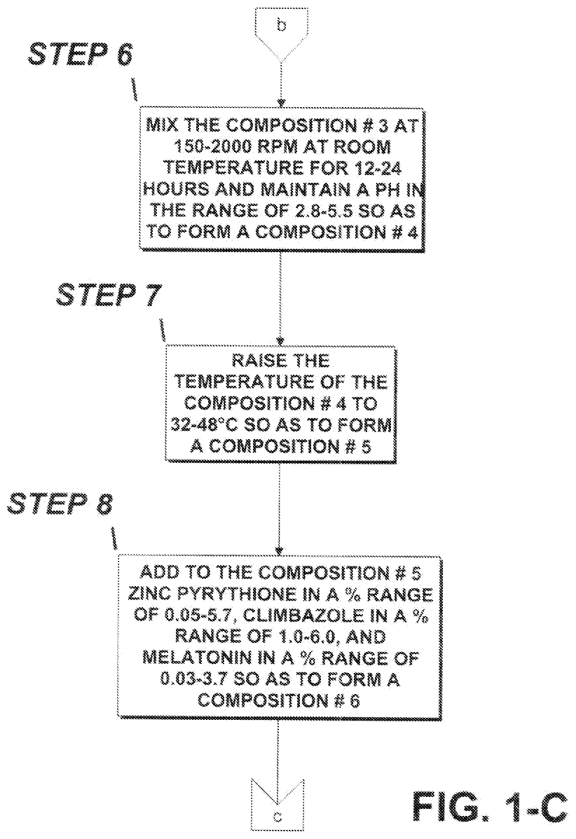

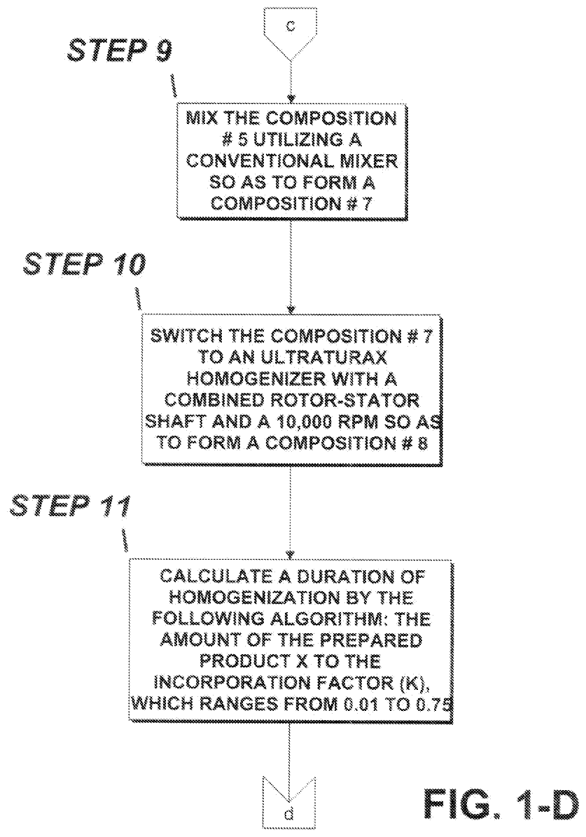

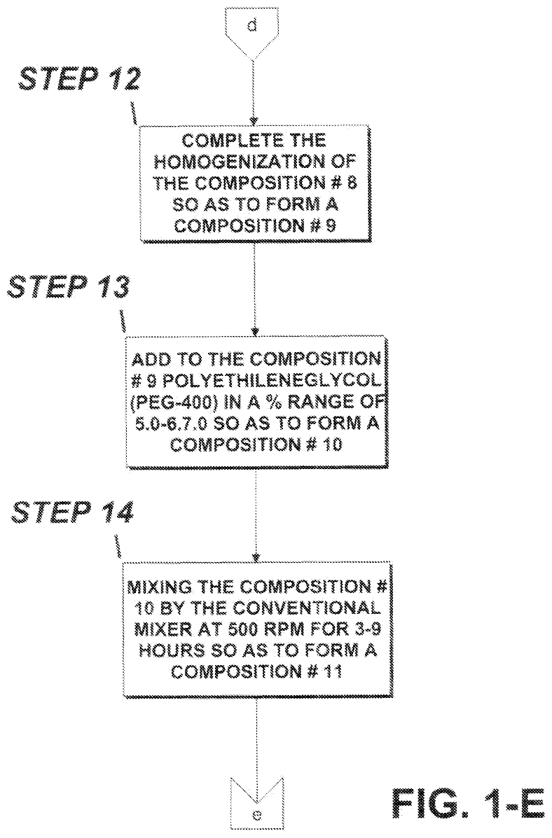

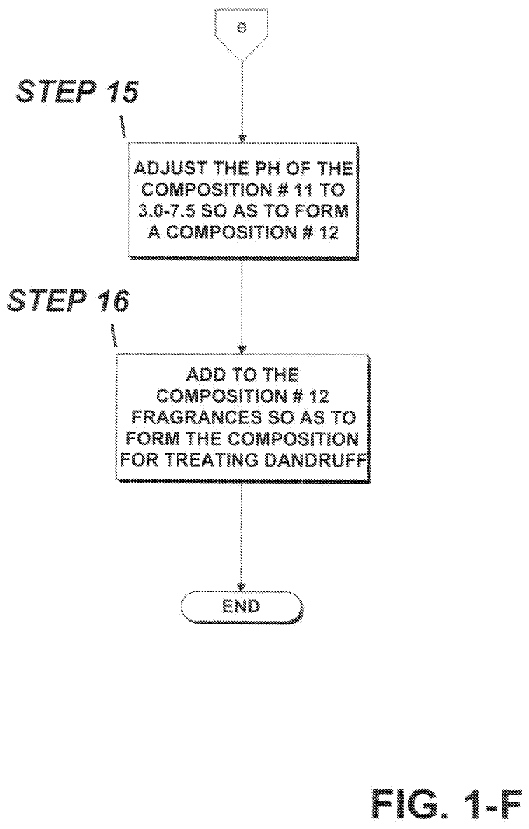

The method of making the composition of the invention shown in an example in FIGS. 1A-1F, and as such, will be discussed with reference thereto. The method shown includes a number of specific amounts and components, but the invention is not exclusively limited to such specific amounts and components, and any reasonable equivalent may be used provided that the key ingredients and steps are retained. The method generally includes the following steps:

The method for making a composition for treating dandruff of the embodiments of the present invention include the steps of: combining a first group of ingredients; stirring the first group of ingredients; combining a second group of ingredients; stirring the second group of ingredients; adding the second group of ingredients to the first group of ingredients and mixing the first and second groups of ingredients together; raising the temperature; adding a third group of ingredients; mixing the first, second, and third groups of ingredients together; switching to a homogenizer (such as for example an Ultraturax (TM) homogenizer); calculating a duration of homogenization; completing the homogenization; adding another ingredient; mixing the first, second, third, and the another ingredient together; adjusting the pH; optionally adding fragrances; and forming the composition for treating dandruff.

Specifically, in an example in FIGS. 1A-1F, the method of making the composition comprises the following steps. All the percentages referred to are expressed as % wt/wt:

STEP1: As shown in FIG. 1A, combine DMSO (Dymethylsulfoxide) in a % range of 4.0-36.0% with Hydroxypropyl-b-clodextrin (HPBC) in a % range of 0.75-27.0 so as to form a composition # 1;

STEP 2: As further shown in FIG. 1A, stir the composition # 1 at room temperature for 2-24 hours using a three-blade propeller shaft at 200-2000 rpm and maintain a pH in the range of 2.0-6.5 so as to form a solution # 1;

STEP 3: As shown in FIG. 1B, combine Distilled Water in a % range of 8.0-46.0, Sodium Chloride in a % range of 0.2-4.3, Potassium Chloride in a % range of 0.7-2.8, Calcium Chloride in a % range of 0.03-5.2, Sodium Bicarbonate in a % range of 0.02-6.1, and Imidazolidynyl Urea in a % range of 0.1-4.5 so as to form a composition # 2;

STEP 4: As further shown in FIG. 1B, stir the composition # 2 at room temperature for 2-12 hours using a three-blade propeller shaft at 100-1500 rpm and maintain a pH in the range of 2.0-5.0 so as to form a solution # 2;

STEP 5: As still further shown in FIG. 1B, add solution # 2 to solution # 1 slowly, so as to form a composition # 3;

STEP 6: As shown in FIG. 1C, mix the composition # 3 at 150-2000 rpm at room temperature for 12-24 hours and maintain a pH in the range of 2.8-5.5 so as to form a composition # 4;

STEP 7: As further shown in FIG. 1C, raise the temperature of the composition # 4 to 32-48° C. so as to form a composition # 5;

STEP 8: As still further shown in FIG. 1C, add to the composition # 5 Zinc Pyrythione in a % range of 0.05-5.7, Climbazole in a % range of 1.0-6.0, and Melatonin in a % range of 0.03-3.7 so as to form a composition # 6;

STEP 9: As shown in FIG. 1D, mix the composition # 5 utilizing a conventional mixer so as to form a composition # 7;

STEP 10: As further shown in FIG. 1D, transfer the composition # 7 to an Ultraturax homogenizer with a combined rotor-stator shaft and a 10,000 rpm so as to form composition # 8;

STEP 11: As still further shown in FIG. 1D, calculate a duration of homogenization by the following algorithm. The duration (min) of Homogenization of/solutions 1 and 2 is calculated based on the final volume of product prepared X to 0.0025. For example, if the final volume is 18600 ml, then the time of Homogenization is: 18600×0.0025=46.5 min.

STEP 12: As shown in FIG. 1E, complete the homogenization of the composition # 8 so as to form a composition # 9;

STEP 13: As further shown in FIG. 1E, add to the composition # 9 Polyethileneglycol (PEG-400) in a % range of 5.0-6.7.0 so as to form composition # 10;

STEP 14: As still further shown in FIG. 1E, mix the composition # 10 by the conventional mixer at 500 rpm for 3-9 hours so as to form composition # 11;

STEP 15: As shown in FIG. 1F, adjust the pH of the composition # 11 to 3.0-7.5 so as to form a composition # 12; and

STEP 16: As further shown in FIG. 1F, add optionally to the composition # 12 fragrances so as to form the composition for treating dandruff.

### TREATMENT FOR DANDRUFF

Another object of the invention is to provide a treatment for dandruff by applying the composition of the invention to a subject in need thereof. Among users of the composition for treating dandruff of the embodiments of the present invention there is an opinion that the composition for treating dandruff of the embodiments of the present invention (or any other hair care topical product) should be applied to a freshly washed head when the hair is still slightly damp. We consider such an approach unjustified. The fact is that moisturizing the skin of the scalp reduces osmolarity, resulting in swelling of the cellular structures of the epidermis. This in turn leads to a narrowing of the intercellular channels and, accordingly, a decrease in the permeability of the scalp. See graph, infra. invention.

Therefore, the composition for treating dandruff of the invention should not be applied to the scalp immediately after washing the head, but should be applied 6-8 hours after washing the head, when the surface of the scalp is dry enough (not moisturized). Alternatively, the composition should be applied after thoroughly drying the scalp with a hair dryer after washing, optionally followed by a delay period of 20-30 minutes before application of the composition. The composition is a leave on application, it is not rinsed out.

The composition and method for treating dandruff encompassed by the present invention are an effective way to control signs of dandruff and seborrheic dermatitis (SD).

For the adult scalp SD, use of the composition for treating dandruff of the embodiments of the present invention formula not only controls the disorder, but also offers the comfort of the complete elimination of any and all symptoms within 30 days.

The composition for treating dandruff of the embodiments of the present invention does not irritate the skin and shows no signs of side effects.

The embodiments of the present invention themselves, however, both as to their construction and to their method of operation, together with additional objects and advantages thereof, will be best understood from the following description of the embodiments of the present invention.

### PRODUCT-BY-PROCESS

For purposes of patentability, the invention includes a product-by-process, being the composition made by the methods described herein. The product-by-process comprises the delivery system and the anti-dandruff biologically active module, made as described, and mixed together to create the formulation product of the invention.

### EXEMPLARY EMBODIMENTS

Specific example of embodiments includes the following.

A formulation for treating and/or preventing dandruff, the formulation comprising the following ingredients: Zinc Pyrythione, Climbazole, Melatonin, Dymethylsulfoxide, Hydroxypropyl-b-Cyclodextrin and water, and further comprising one or more compounds selected from of the group consisting of Sodium Chloride, Potassium Chloride, Calcium Chloride, Sodium Bicarbonate and Imidazolidynyl Urea.

In this formulation the ingredients may be present in the following % wt/wt. Zinc Pyrythione: 0.05-5.7%, Climbazole: 1.0-6.0%, Melatonin: 0.03-3.7%, Dymethylsulfoxide: 4.0-36.0%, Hydroxypropyl-b-Cyclodextrin: 0-27.0%, Sodium Chloride: 0-4.3%, Potassium Chloride: 0-2.8%, Calcium Chloride: 0-5.2%, Sodium Bicarbonate: 0-6.1%, Imidazolidynyl Urea: 0-4.5, and Water: to 100% vol or 100% wt.

Alternatively, in this formulation the ingredients may be present in the following % wt/wt. The formulation of claim 1 wherein the ingredients are present in the following percentage ranges % wt/wt Zinc Pyrythione: 0.05-5.7%, Climbazole: 1.0-6.0%, Melatonin: 0.03-3.7%, Dymethylsulfoxide: 4.0-36.0%, Hydroxypropyl-b-Cyclodextrin: 0.75-27.0%, Sodium Chloride: 0.2-4.3%, Potassium Chloride: 0.7-2.8%, Calcium Chloride: 0.03-5.2%, Sodium Bicarbonate: 0.02-6.1%, Imidazolidynyl Urea: 0.1-4.5, and Water: 8.0-46.0%.

Alternatively, in this formulation the ingredients may be present without any other compounds being present (“consisting of” the above).

The invention also encompasses a method for making a formulation for treating and preventing dandruff, the method comprising the following steps:

(1) in a first vessel, combine DMSO with Hydroxypropyl-b-clodextrin and stir the composition at room temperature for 2-24 hours while maintaining maintain pH between 2.0 and 6.5; (2) in a second vessel, add Sodium Chloride, Potassium Chloride, Calcium Chloride, Sodium Bicarbonate and Imidazolidynyl Urea to water and stir at room temperature for 2-12 hours while maintaining pH between 2.0-5.0; (3) slowly add the contents of the second vessel to the contents of the first vessel, and mix the resulting mixture at room temperature for 12-24 hours while maintaining pH between 2.8-5.5; (4) raise the temperature of the mixture to between 32° C. to 48° C., then add Zinc Pyrythione, Climbazole and Melatonin to the mixture and continue to mix; (5) homogenize using a high-speed homogenizer until the mixture is homogeneous; (6) add PEG and continue mixing for 3 to 9 hours, then adjust the pH to between 3.0-7.5 whereby the resultant composition is a formulation for use for treating dandruff.

In an alternate embodiment in step (1) stirring for 2-24 is done at a stirring speed between 200 and 2000 RPM; In step (2) stirring for 2-12 is done at a stirring speed between 100 and 1500 RPM; In step (3) stirring for 12-24 is done at a stirring speed between 150 and 2000 RPM; and in step (5) homogenization is done at a stirring speed between 7000 and 12000 RPM.

In one embodiment Zinc Pyrythione is added to produce a final concentration in the formulation of 0.05-5.7% wt/wt %, Climbazole Pyrythione is added to produce a final concentration in the formulation of 1.0-6.0 wt/wt %, and Melatonin Pyrythione is added to produce a final concentration in the formulation of 0.03-3.7% wt/wt.

Another embodiment encompasses a product-by-process for treating and preventing dandruff, made by the following process:(1) in a first vessel, combine DMSO with Hydroxypropyl-b-clodextrin and stir the composition at room temperature for 2-24 hours while maintaining maintain pH between 2.0 and 6.5; (2) in a second vessel, add Sodium Chloride, Potassium Chloride, Calcium Chloride, Sodium Bicarbonate and Imidazolidynyl Urea to water and stir at room temperature for 2-12 hours while maintaining pH between 2.0-5.0; (3) slowly add the contents of the second vessel to the contents of the first vessel, and mix the resulting mixture at room temperature for 12-24 hours while maintaining pH between 2.8-5.5; (4) raise the temperature of the mixture to between 32° C. to 48° C., then add Zinc Pyrythione, Climbazole and Melatonin to the mixture and continue to mix; (5) homogenize using a high-speed homogenizer until the mixture is homogeneous; and (6) add PEG and continue mixing for 3 to 9 hours, then adjust the pH to between 3.0-7.5 whereby the resultant composition is a formulation for use for treating dandruff. In one other embodiment, in step (1) stirring for 2-24 is done at a stirring speed between 200 and 2000 RPM; In step (2) stirring for 2-12 is done at a stirring speed between 100 and 1500 RPM; In step (3) stirring for 12-24 is done at a stirring speed between 150 and 2000 RPM; and in step (5) homogenization is done at a stirring speed between 7000 and 12000 RPM. The product-by-process may include wherein: Zinc Pyrythione is added to produce a final concentration in the formulation of 0.05-5.7% wt/wt %, Climbazole Pyrythione is added to produce a final concentration in the formulation of 1.0-6.0 wt/wt %, and Melatonin Pyrythione is added to produce a final concentration in the formulation of 0.03-3.7 wt/wt %.

Another embodiment encompasses a method of treating or preventing dandruff, the method comprising: (1) providing a formulation for treating and preventing dandruff, the formulation comprising the following ingredients Zinc Pyrythione, Climbazole, Melatonin, Dymethylsulfoxide, Hydroxypropyl-b-Cyclodextrin and water, and further comprising one or more compounds selected from of the group consisting of Sodium Chloride, Potassium Chloride, Calcium Chloride, Sodium Bicarbonate and Imidazolidynyl Urea; and (2) applying the formulation to the scalp of a subject in need of such treatment wherein such application is not done immediately after washing the scalp, but is done when the scalp is dry. In this method application of the formulation to the scalp is done at least 20 minutes after washing the scalp, when the scalp is thoroughly dry and when osmolarity of the skin of the scalp is low.

### GENERAL DISCLOSURES

This specification incorporates by reference all documents referred to herein and all documents filed concurrently with this specification or filed previously in connection with this application, including but not limited to such documents which are open to public inspection with this specification. All numerical quantities mentioned herein include quantities that may be plus or minus 20% of the stated amount in every case, including where percentages are mentioned. As used in this specification, the singular forms “a, an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. For example, a composition “comprising” (or “which comprises”) ingredients A, B and C can contain only ingredients A, B and C, or can contain not only ingredients A, B and C but also one or more other ingredients. The term “consisting essentially of” and grammatical equivalents thereof is used herein to mean that, in addition to the features specifically identified, other features may be present which do not materially alter the claimed invention. The term “at least” followed by a number is used herein to denote the start of a range beginning with that number (which may be a range having an upper limit or no upper limit, depending on the variable being defined). For example, “at least 1” means 1 or more than 1, and “at least 80%” means 80% or more than 80%. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). When, in this specification, a range is given as “(a first number) to (a second number)” or “(a first number)-(a second number)”, this means a range whose lower limit is the first number and whose upper limit is the second number. For example, “from 40 to 70 microns” or “40-70 microns ” means a range whose lower limit is 40 microns, and whose upper limit is 70 microns.

When discussing the amount of a compound in a final LIQUID formulation, we may use the relative units of Percent Volume (v/v). Typically, percent volume written as ‘% Vol’ or ‘Volume percent’ or ‘volume/volume percent’ or ‘v/v %’. This term is used when preparing solutions of liquids. Volume percent is defined as: v/v %=[(volume of solute)/(volume of solution)]×100%. Note that volume percent is relative to the volume of solution, not the volume of solvent. For example, wine is about 12% v/v ethanol. This means there is 12 ml ethanol for every 100 ml of wine. It is important to realize liquid and gas volumes are not necessarily additive. If you mix 12 ml of ethanol and 100 ml of wine, you will get less than 112 ml of solution.

Alternatively, in a LIQUID formulation, quantities can be given in terms of relative per cent concentration BY WEIGHT OF SOLUTE in a solution by volume (sometimes given as the relative units wt/vol % or % wt/vol or wt/vol or wt/v or w/v). To determine the weight per cent of a solution, divide the mass of solute by mass of the solution (solute and solvent together) and multiply by 100 to obtain per cent. The per cent by weight formula=gram of solute/100 g of solution.

Quantities in a SOLID formulation are given wt/wt %, also called weight fraction, sometimes shown as wt/wt or % wt/wt or wt/wt %.

Alternatively, and as frequently used in this disclosure, any mixtures such as solids in liquids, liquid in liquid and solids mixed with solids can also be expressed as % wt/wt. The weight of water is generally about 1 g/ml, so wt/vol and wt/wt is often similar or the same for a generally aqueous solution.

### IMPRESSIONS

It will be understood that each of the elements described above, or two or more together, may also find a useful application in other types of constructions differing from the types described above. While the embodiments of the present invention have been illustrated and described as embodied in a method of making a composition for treating dandruff, nevertheless, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention. This disclosure foregoing will so fully reveal the gist of the embodiments of the present invention that others can by applying current knowledge readily adapt them for various applications without omitting features from the standpoint of prior art fairly constitute characteristics of the generic or specific aspects of the embodiments of the present invention.

1. A formulation for treating and preventing dandruff, the formulation comprising the following ingredients:
Zinc Pyrythione,Climbazole,Melatonin,Dymethylsulfoxide,Hydroxypropyl-b-Cyclodextrin and water, and further comprising one or more compounds selected from of the group consisting of Sodium Chloride, Potassium Chloride, Calcium Chloride, Sodium Bicarbonate and Imidazolidynyl Urea.

2. A formulation claim 1 comprising the following ingredients Zinc Pyrythione, Climbazole, Melatonin, Dymethylsulfoxide, Hydroxypropyl-b-Cyclodextrin and water, and further comprising Sodium Chloride, Potassium Chloride, Calcium Chloride, Sodium Bicarbonate and Imidazolidynyl Urea.

3. The formulation of claim 1 wherein the ingredients are present in the following percentages (% wt/wt):
Zinc Pyrythione: 0.05-5.7%
Climbazole: 1.0-6.0%
Melatonin: 0.03-3.7%
Dymethylsulfoxide: 4.0-36.0%
Hydroxypropyl-b-Cyclodextrin: 0-27.0%
Sodium Chloride: 0-4.3%
Potassium Chloride: 0-2.8%
Calcium Chloride: 0-5.2%
Sodium Bicarbonate: 0-6.1%
Imidazolidynyl Urea: 0-4.5%, and
Water: to 100%.

4. The formulation of claim 1 wherein the ingredients are present in the following percentage ranges (% wt/wt):
Zinc Pyrythione: 0.05-5.7%
Climbazole: 1.0-6.0%
Melatonin: 0.03-3.7%
Dymethylsulfoxide: 4.0-36.0%
Hydroxypropyl-b-Cyclodextrin: 0.75-27.0%
Sodium Chloride: 0.2-4.3%
Potassium Chloride: 0.7-2.8%
Calcium Chloride: 0.03-5.2%
Sodium Bicarbonate: 0.02-6.1%
Imidazolidynyl Urea: 0.1-4.5, and
Water: 8.0-46.0%.

5. The formulation of claim 1 consisting of:
Zinc Pyrythione,
Climbazole,
Melatonin,
Dymethylsulfoxide,
Hydroxypropyl-b-Cyclodextrin,
Sodium Chloride,
Potassium Chloride,
Calcium Chloride,
Sodium Bicarbonate,
Imidazolidynyl Urea and water.

6. A method for making a formulation for treating and preventing dandruff, the method comprising the following steps:
(1) in a first vessel, combine DMSO with Hydroxypropyl-b-clodextrin and stir the composition at room temperature for 2-24 hours while maintaining pH between 2.0 and 6.5;
(2) in a second vessel, add Sodium Chloride, Potassium Chloride, Calcium Chloride, Sodium Bicarbonate and Imidazolidynyl Urea to water and stir at room temperature for 2-12 hours while maintaining pH between 2.0-5.0;
(3) slowly add the contents of the second vessel to the contents of the first vessel, and mix the resulting mixture at room temperature for 12-24 hours while maintaining pH between 2.8-5.5;
(4) raise the temperature of the mixture to between 32° C. to 48° C., then add Zinc Pyrythione, Climbazole and Melatonin to the mixture and continue to mix;
(5) homogenize using a high-speed homogenizer until the mixture is homogeneous;
(6) add PEG and continue mixing for 3 to 9 hours, then adjust the pH to between 3.0-7.5 whereby the resultant composition is a formulation for use for treating dandruff.

7. The method of claim 6 wherein:
In step (1) stirring for 2-24 is done at a stirring speed between 200 and 2000 RPM;
In step (2) stirring for 2-12 is done at a stirring speed between 100 and 1500 RPM;
In step (3) stirring for 12-24 is done at a stirring speed between 150 and 2000 RPM; and
In step (5) homogenization is done at a stirring speed between 7000 and 12000 RPM.

8. The method of claim 6 wherein Zinc Pyrythione is added to produce a final concentration in the formulation of 0.05-5.7% wt/wt, Climbazole Pyrythione is added to produce a final concentration in the formulation of 1.0-6.0% wt/wt, and MelatoninPyrythione is added to produce a final concentration in the formulation of 0.03-3.7 5 wt/wt.

9. A product-by-process for treating and preventing dandruff, made by the following process:
(1) in a first vessel, combine DMSO with Hydroxypropyl-b-clodextrin and stir the composition at room temperature for 2-24 hours while maintaining pH between 2.0 and 6.5;
(2) in a second vessel, add Sodium Chloride, Potassium Chloride, Calcium Chloride, Sodium Bicarbonate and Imidazolidynyl Urea to water and stir at room temperature for 2-12 hours while maintaining pH between 2.0-5.0;
(3) slowly add the contents of the second vessel to the contents of the first vessel, and mix the resulting mixture at room temperature for 12-24 hours while maintaining pH between 2.8-5.5;
(4) raise the temperature of the mixture to between 32° C. to 48° C., then add Zinc Pyrythione, Climbazole and Melatonin to the mixture and continue to mix;
(5) homogenize using a high-speed homogenizer until the mixture is homogeneous; and
(6) add PEG and continue mixing for 3 to 9 hours, then adjust the pH to between 3.0-7.5 whereby the resultant composition is a formulation for use for treating dandruff.

10. The product-by-process of claim 9 wherein:
in step (1) stirring for 2-24 is done at a stirring speed between 200 and 2000 RPM;
in step (2) stirring for 2-12 is done at a stirring speed between 100 and 1500 RPM;
in step (3) stirring for 12-24 is done at a stirring speed between 150 and 2000 RPM; and
in step (5) homogenization is done at a stirring speed between 7000 and 12000 RPM;

11. The product-by-process of claim 9 wherein Zinc Pyrythione is added to produce a final concentration in the formulation of 0.05-5.7% wt/wt, Climbazole Pyrythione is added to produce a final concentration in the formulation of 1.0-6.0% wt/wt, and Melatonin Pyrythione is added to produce a final concentration in the formulation of 0.03-3.7% wt/wt.

12. A method of treating or preventing dandruff, the method comprising:
(1) providing a formulation for treating and preventing dandruff, the formulation comprising the following ingredients: Zinc Pyrythione, Climbazole, Melatonin, Dymethylsulfoxide, Hydroxypropyl-b-Cyclodextrin and water, and further comprising one or more compounds selected from of the group consisting of Sodium Chloride, Potassium Chloride, Calcium Chloride, Sodium Bicarbonate and Imidazolidynyl Urea; and
(2) applying the formulation to the scalp of a subject in need of such treatment wherein such application is not done immediately after washing the scalp, but is done when the scalp is dry and is a leave on application that is not rinsed out.

13. The method of claim 12 wherein said application of the formulation to the scalp is done at least 20 minutes after washing the scalp, when the scalp is thoroughly dry and when osmolarity of the skin of the scalp is low.
